(19) United States
(12) Patent Application Publication
(10) Pub. No.: US 2021/0137684 A1
(43) Pub. Date: May 13, 2021

(54) POST-COATING MACHINING

(71) Applicant: Spinal Elements, Inc., Carlsbad, CA (US)

(72) Inventors: Dean Johnson, Solana Beach, CA (US); Chris Morris, Encinitas, CA (US)

(73) Assignee: Spinal Elements, Inc., Carlsbad, CA (US)

(21) Appl. No.: 17/086,689

(22) Filed: Nov. 2, 2020

Related U.S. Application Data
(60) Provisional application No. 62/932,693, filed on Nov. 8, 2019.

(51) Int. Cl. A61F 2/30; B05D 3/12

(52) U.S. Cl. CPC A61F 2/3094; B05D 3/12; A61F 2/4455; A61F 2002/30978; A61F 2002/30985; A61F 2002/3097

(57) ABSTRACT

A method of manufacturing an intervertebral implant comprising forming the implant with at least one dimension that is greater than a desired dimension, the implant comprising a superior bone facing surface, an inferior bone facing surface, a distal side, a proximal side, and lateral sides; coating at least the superior bone facing surface and the inferior bone facing surface with an osteophilic material; and machining one or more of the distal side, proximal side and lateral sides to the desired dimensions after coating; wherein edges of the coating on the superior bone facing surface and the inferior bone facing surface are machined to be flush with the distal side, proximal side and/or lateral sides.

### CROSS-REFERENCE TO RELATED APPLICATIONS

This application claims priority benefit to U.S. Provisional Patent Application No. 62/932,693, filed Nov. 8, 2019, which is incorporated herein by reference in its entirety for all purposes.

### FIELD

The present application relates generally to processes for preparing a medical device, and more particularly to methods for manufacturing spinal implants that are coated with fusion promoting material.

### BACKGROUND

The spine is a flexible structure that extends from the base of the skull to the tailbone. The weight of the upper body is transferred through the spine to the hips and the legs. The spine contains a plurality of bones called vertebrae. The vertebrae are hollow and stacked one upon the other, forming a strong hollow column for support. The hollow core of the spine houses and protects the nerves of the spinal cord. Each vertebra is separated from the vertebra above or below by a cushion-like, fibrocartilage called an intervertebral disc. The discs act as shock absorbers, cushioning the spine, and preventing individual bones from contacting each other. In addition, intervertebral discs act as a ligament that holds vertebrae together. Intervertebral discs also work with the facet joint to allow for slight movement of the spine. Together, these structures allow the spine to bend, rotate and twist.

The spinal structure can become damaged as a result of degeneration, dysfunction, disease and or trauma. More specifically, the spine may exhibit disc collapse, abnormal curvature, asymmetrical disc space collapse, abnormal alignment of the vertebrae and general deformity, which may lead to imbalance and tilt in the vertebrae. This may result in nerve compression, disability and overall instability and pain. If the proper shaping or curvature are not present due to scoliosis, neuromuscular disease, cerebral palsy, or other disorder, it may be necessary to straighten or adjust the spine into a proper curvature with surgery to correct these spinal disorders.

Some ailments of the spine result in degeneration of the spinal disc in the intervertebral space between adjacent vertebrae. Disc degeneration can cause pain and other complications. Conservative treatment can include non-operative treatment requiring patients to adjust their lifestyles and submit to pain relievers and a level of underlying pain. Operative treatment options include disc removal. This can relieve pain in the short term, but also often increases the risk of long-term problems and can result in motor and sensory deficiencies resulting from the surgery. Disc removal and more generally disc degeneration disease are likely to lead to a need for surgical treatment in subsequent years. The fusion or fixation of vertebrae will minimize or substantially eliminate relative motion between the fixed or fused vertebrae. In surgical treatments, interbody implants may be used to correct disc space collapse between adjacent vertebrae, resulting in spinal fusion of the adjacent vertebrae.

A fusion is a surgical method wherein two or more vertebrae are joined together (fused) by way of interbody implants, sometimes with bone grafting, to form a single bone. The current standard of care for interbody fusion requires surgical removal of all or a portion of the intervertebral disc. After removal of the intervertebral disc, the interbody implant is implanted in the interspace. In many cases, the fusion is augmented by a process called fixation. Fixation refers to the placement of screws, rods, plates, or cages to stabilize the vertebrae so that fusion can be achieved.

### SUMMARY

A method of manufacturing an intervertebral implant is provided. The method can include forming the implant with at least one dimension that is greater than a desired dimension. The implant can include a superior bone facing surface, an inferior bone facing surface, a distal side, a proximal side, and lateral sides. The method can include coating at least the superior bone facing surface and the inferior bone facing surface with an osteophilic material. The method can include machining one or more of the distal side, proximal side and lateral sides to the desired dimensions after coating, wherein edges of the coating on the superior bone facing surface and the inferior bone facing surface are machined to be flush with the distal side, proximal side and lateral sides.

A method of manufacturing an orthopedic implant is provided. The method can include forming a part with at least one dimension that is greater than a desired dimension. The method can include coating at least a portion of the part with an osteophilic material. The method can include machining the part to the desired dimension after coating. The method can include machining portions of the coating. The coating can be machined to be flush with a side of the part. The part can be machined to the desired dimension by one or more of milling, drilling, laser cutting, electrical discharge machining, sanding, and filing. The osteophilic material is one of the group consisting of titanium, titanium alloy, cobalt-chrome, stainless steel, hydroxylapatite, and allograft. The method can include assembling the part with a second part after machining. The method can include assembling the part with a second part before machining. The part can be formed by one or more of 3-D printing, molding, machining, casting, and extruding. The part can be not masked before the coating step. Machining the part can include machining a tool engagement side of the implant.

A method of manufacturing an orthopedic implant can include forming a first part with at least one dimension that is greater than a desired dimension. The method can include coating at least a portion of the first part with an osteophilic material. The method can include machining the first part to the desired dimension after coating. The method can include forming a second part with at least one dimension that is greater than a desired dimension. The method can include coating at least a portion of the second part with an osteophilic material. The method can include machining the second part to the desired dimension after coating. The method can include assembling together the first part and the second part. The orthopedic implant can be an expandable intervertebral device. The first part can be a top plate that is coated on a superior bone facing side and the second part can be a bottom plate that is coated on an inferior bone facing side. Machining the first part or second part to the desired dimension can include machining portions of the coating. The coating can be machined to be flush with a side of the first part or second part. The first part and the second part can be machined to the desired dimension by one or more of milling, drilling, laser cutting, electrical discharge machining, sanding, and filing. The osteophilic material is one of the group consisting of titanium, titanium alloy, cobalt-chrome, stainless steel, hydroxylapatite, and allograft. The first part and the second part can be formed by one or more of 3-D printing, molding, machining, casting, and extruding. The first part and the second part can be not masked before the coating step.

### BRIEF DESCRIPTION OF THE DRAWINGS

These and other features, aspects and advantages of the described embodiments are described with reference to drawings of certain embodiments, which are intended to illustrate, but not to limit. It is to be understood that the attached drawings are for the purpose of illustrating concepts of the described embodiments and may not be to scale.

FIG. 1 is a flowchart illustrating the steps of producing a coated implant, according to the prior art.

FIG. 2 is a front perspective view of a coated implant made in accordance with the method of FIG. 1.

FIG. 3 is a back perspective view of a coated implant made in accordance with the method of FIG. 1.

FIG. 4 is a side view of a coated implant made in accordance with the method of FIG. 1.

FIG. 5 is a bottom view of a coated implant made in accordance with the method of FIG. 1.

FIG. 6 is a flowchart illustrating the steps of producing a coated implant, according to an embodiment of the present disclosure.

FIG. 7 is a front perspective view of a coated implant made in accordance with the method of FIG. 6.

FIG. 8 is a back perspective view of a coated implant made in accordance with the method of FIG. 6.

FIG. 9 is a side view of a coated implant made in accordance with the method of FIG. 6.

FIG. 10 is a bottom view of a coated implant made in accordance with the method of FIG. 6.

### DETAILED DESCRIPTION

Portions of some interbody implants can be coated with an osteophilic material to promote integration of the implant with the surrounding bones and tissue at the implant site of a patient. For example, the implant can include a titanium or titanium alloy coating, particularly on the surfaces of the implant that contact the patient's bone and tissue. Osteophilic coatings have been shown to provide faster and improved osseointegration of the implant with the native bone and tissue. Other coating material can be used, such as cobalt-chrome, stainless steel, hydroxylapatite, allograft and the like. The coating material can be applied to the implant through any of a plurality of methods, such as rubbing, spraying, dipping, painting and the like.

The coating can have a porous or roughened surface that helps with osteointegration between the implant and bone/tissue. The porous or roughened surface can provide a good platform for growth of bone and tissue. Any of a plurality of different methods can be used to achieve a porous or roughened surface, such as spray depositing the coating onto the implant. Other methods of producing a roughened coating are also contemplated, including media blasting the coating or acid bath treatment of the coating.

FIG. 1 is a flowchart showing an example of a previously existing procedure for producing a coated implant. First, the part is completely machined or substantially completely machined 10, in which the part is 90% or more completely machined. The part can be made from materials such as PEEK, cobalt chrome or stainless steel. Next, the implant is assembled 12, if assembly is required. Assembly can comprise connecting two or more components of the implant together with adhesives, fasteners, pins, and the like. In some embodiments, differently sized and/or shaped components can be assembled with another base component to customize the implant for different patients. At this point in the procedure, the implant is substantially complete and similar to non-coated implants.

The implant is inspected for any defects or dimensions that are beyond acceptable limits 14. For instance, a critical portion can be measured to see if it is within design tolerances. In another example, the walls, struts, and other structural components of the machined part can be tested to determine if they meet specified load ratings. If the implant is determined to be defective or out of specification, the implant is further inspected to determine if it can be repaired by additional machining or other adjustment 24. If the implant cannot be repaired, then it is discarded or recycled 26.

If the machined implant is acceptable, the implant can be prepared for the coating process. The coating process can be labor and time intensive. In some embodiments, portions of the implants are masked before coating 16 to protect the parts of the implant where the coating is not desired. For example, the fastener holes of the implant can be masked prior to coating so that the coating is not applied to these surfaces. Coating the fastener holes can lead to improper securement of the fasteners and reduced implanted stability and can compromise fusion. In some extreme situations, improperly coated implants can cause one or more of fastener to back out and lead to implant failure. The large amount of time and labor needed to apply the masking to select portions of the implant can increase manufacturing costs and can also lead to human error in the masking process. Other disadvantages of masking the implant before coating are discussed below.

In some embodiments, the implant is media blasted 18 prior to coating to remove contaminants from the implant and prepare the surfaces to accept the coating material. In some embodiments, the implant surfaces are treated with acid or other solution to prepare the surface for coating. Then, the implant is coated 20 with an osteophilic material to promote integration of the implant with the surrounding bones and tissue when implanted, such as for example titanium or titanium alloy coating. A thin coating can be applied just enough to cover the surface of the implant, or the coating can be thicker and have a thickness of several millimeters.

In some situations, portions of the implant that are structurally fragile may break during the coating process. The heat, impact, harsh chemicals and other stresses from the coating process can fracture or weaken structural components of the implant. For example, a thin cross-member of the implant can fracture when the coating is sprayed onto the implant. In another example, the heat from the coating may deform portions of the implant having critical dimensions to the point where the deformed portions are no longer within acceptable dimensional tolerances.

After coating, the implant can be post-coating media blasted 22 to remove contaminants from the implant and prepare the surfaces to enhance osteointegration between the implant and bone/tissue. The masking can be removed 28, which is a labor intensive and time consuming process. In some situations, the coating can harden and make the removal of the masking difficult. The removal of the masking can also damage the coating and leave uneven edges, as illustrated for example in FIGS. 2-5.

The implant can be sanded and/or filed 30. The sanding and/or filing can include detailed adjustment of the implant, such as removing coating ingress under the masked portions. The implant can be washed and cleaned in preparation for final inspection.

The implant and coating are inspected 32 for any defects, damage, warping or other unacceptable conditions. Inspection can involve one or more of visual inspections, structural load tests, imaging (e.g., ultrasonic, x-ray, infrared, etc.), measurements of select dimensions, and the like. If the implant is determined to be defective or otherwise unacceptable, the implant is inspected to determine if it can be repaired by additional machining or other adjustment 24. If the implant cannot be repaired, then it is discarded or recycled 26. Since the coating procedure is performed after complete machining of the implant, any implants that are damaged during the coating process results in a loss of money and time spent in the machining, masking and coating of the implant.

If the implant passes inspection, then the coated implant can be cleaned/sterilized 34 and packaged 36 for storage and transport. Methods of cleaning and sterilization can include autoclaving, rinsing with a cleaning solution, ultrasonic bath, and the like. The finished implant can be packaged in custom containers that secure the implant in a protective package that protects the implant from forces and impacts experienced during transport and storage. In some embodiments, the packaging seals the implant in a sterilized environment (e.g., hermetically sealed) to maintain the sterilization of the implant.

### New Procedure

An aspect of at least one of the embodiments disclosed herein includes the realization that there remains a need for methods of manufacturing a coated implant that minimize the time, human error and manufacturing scrap rate associated with coating procedures while increasing the quality of the finished coated implant. The following disclosure describes improved spinal implants and methods of manufacturing the implants for use in the immobilization and fusion of orthopedic joints.

A method of manufacturing the coated interbody device can include a post-coating machining procedure. The coating can be applied to the implant through any of a variety of different coating methods. The implant can then be machined after the coating is applied, removing the coating from the portions of the implant where the coating is not desired.

A detailed description of the post-coating machining procedure will be described with reference to FIG. 6. First, the part can be formed 110 through 3-D printing, molding, machining, casting, and the like. The initial formation of the part can have rough dimensions and large tolerances on at least some portions, saving time and cost in the formation step. In some embodiments, the initial formation of the part comprises a shaped block of material with or without engagement features, such as teeth or ridges, on a superior and/or inferior portion of the part. In some embodiments, the initial formation of the implant comprises a portion or dimension of the implant that is greater than the portion or dimension desired as a finished implant. In some embodiments, engagement features, such as teeth or ridges, are machined on a portion of the superior and/or inferior surface of the implant, such that the post-coating machining of the implant will remove at least a portion of the perimeter outside of the engagement features. In some instances, removing at least a portion of the perimeter outside of the engagement features allows for the machined surface of the coating to be flush with a non-coated surface, whereas removing a portion of the perimeter at or within the portion comprising the engagement features can create non-contiguous or uneven portions of coating or cause the coating to degrade or fracture at the boundary between the coated surface and a non-coated surface. Such erratic border edges of the coating material can lead to areas of undesirable fusion and bone growth. In some embodiments, however, the portions that are to be covered with coating can be made with greater accuracy than other portions that are to be machined later. The part can be made from any biocompatible material, such as for example PEEK, cobalt chrome or stainless steel.

Next, the part can be assembled 112, if assembly is required. In some embodiments, a first component and a second component can be formed separately and assembled together with adhesives, fasteners, pins, and the like. For example, a left component and a right component can be machined separately and then assembled to form intricate features in the middle of the assembled implant that would otherwise not be machinable in a single component.

The implant is inspected 114 for any defects or dimensions that are beyond acceptable limits. Inspection can involve one or more of visual inspections, structural load tests, imaging (e.g., ultrasonic, x-ray, infrared, etc.), measurements of select dimensions, and the like. If the implant is determined to be defective or out of specification, the implant is further inspected to determine if the part can be repaired by additional processing or other adjustment 122. If the implant cannot be repaired, then it is discarded or recycled 124.

If the formed part is acceptable, the part can be prepared for the coating process. The part can be coated without any masking of the part. In some situations, minimal masking is performed before coating to protect certain parts of the implant, such as portions where subsequent machining is difficult or not possible (e.g., undercuts). In some situations, some masking can be performed, but without the same amount of care and precision as in current coating methods, since subsequent machining will adjust the coating to the precise dimensions.

In some embodiments, the part is media blasted 116 prior to coating to remove contaminants from the part and prepare the surfaces to accept the coating material. In some embodiments, the part surfaces are treated with acid or other solution to prepare the surfaces for coating. Then, the part is coated 118 with an osteophilic material to promote integration of the implant with the surrounding bones and tissue when implanted, such as titanium or titanium alloy coating. A thin coating can be applied just enough to cover the surface of the implant, or the coating can be thicker and have a thickness of several millimeters. After coating, the part can be post-coating media blasted 120 again to remove contaminants from the part and treat the surfaces to enhance osteointegration between the implant and bone/tissue.

The implant and coating can be inspected 126 for any defects, damage, warping or other unacceptable conditions. Inspection can involve one or more of visual inspections, structural load tests, imaging (e.g., ultrasonic, x-ray, infrared, etc.), measurements of select dimensions, and the like. If the implant is determined to be defective or otherwise unacceptable, the implant is inspected to determine if it can be repaired or otherwise adjusted 122. If the implant cannot be repaired, then it is discarded or recycled 124. Since the coating procedure is performed before machining, if the part is damaged during the coating process then any loss of money and time expended on a discarded part is limited to the initial forming and coating of the part.

If the implant passes inspection, then the coated part can be machined to the desired specifications 128. The machining process can include, but is not limited to, fabrication methods such as milling, drilling, deburring, laser cutting, electrical discharge machining (EDM), sanding, filing, and the like. As described in detail below, the post-coating machining method has several advantages over previous coating methods. Some advantages include faster production times, reduced scrap rate and costs, more precise dimensions, less production errors and improved aesthetics.

In embodiments where the part is masked, the masking can be removed and any final sanding or filing can be performed on the implant. The final sanding or filing can include detailed adjustment of the implant, such as cropping excessively coated areas.

The implant and coating can be inspected 130 again for any defects, damage, warping or other unacceptable conditions. If the implant is determined to be defective or otherwise unacceptable, the implant is examined to determine if it can be repaired by additional machining or other adjustment 122. If the implant cannot be repaired, then it is discarded or recycled 124.

In some embodiments, the implant includes more than one part that are coated separately and then assembled. The parts can be assembled before the machining step or after the machining step. For example, an expandable intervertebral implant can have a top plate and a bottom plate that are coated on some surfaces, such as the superior and inferior bone facing sides. The top plate and bottom plate can be assembled and then machined to the final dimensions, which also cleans the non-coating surfaces of coating overspray. In another example, an expandable intervertebral implant includes a top plate and a bottom plate that are coated and then machined separately. The top plate and bottom plate can then be assembled together.

Once the implant has been inspected to ensure that it conforms to specifications, the coated implant can be cleaned/sterilized 132 and packaged 134 for storage and transport. Methods of cleaning and sterilization can include autoclaving, rinsing with a cleaning solution, ultrasonic bath, and the like. The finished implant can be packaged in custom containers that secure the implant in a protective package that protects the implant from forces and impacts experienced during transport and storage. In some embodiments, the packaging seals the implant in a sterilized environment (e.g., hermetically sealed) to maintain the sterilization of the implant.

### Advantages

The post-coating machining method disclosed herein has several advantages over previous methods of coating after machining the implant. An advantage of the post-coating machining method is faster production times compared to previous methods. One of the contributing factors of the faster production times is the elimination or reduction of the masking step. Previous methods involved masking portions of a machined implant before the coating process, which can be time consuming and expensive. The masking process is typically manually performed by a person carefully placing masking tape on the areas of the implant where coating is not desired. For example, the side surfaces of an implant can be masked so that only the top and bottom surfaces of the implant are coated with material. In some situations, the shape and contours of the implant may be complex and/or the coating boundaries can be intricate, such that the masking needs to be applied carefully, which can be time consuming. In some situations, the implant is small in size and the masking is delicate and can be difficult to apply. In some situations, silicone masking is applied, which requires tooling and production to prepare a suitable silicone mask, which may leave residue, such as silicone or adhesives, or other contaminants on or within the implant.

In the post-coating machining method, the masking step can be eliminated or reduced because the machining process can remove at least some of the overspray of coating material. For example, with reference to FIG. 7, when coating the top 156 of the implant 150, some of the coating 162 material may be inadvertently applied to some adjacent surfaces, such as the front 152 of the implant 150. The unwanted coating material on the front 152 is removed when the front 152 is machined, leaving an uncoated face with a clean edge 164 of coating material, as illustrated in FIG. 7. Eliminating or reducing the masking of the implant prior to the coating procedure advantageously decreases the manufacturing production times. In addition, the post-coating machining method eliminates or reduces the introduction of residue, such as silicone or adhesives, or other contaminants on or within the implant, such as from silicone, for example.

Another advantage of the post-coating machining method is reduced human error. As mentioned above, the masking can be complex and intricate, which can lead to errors by the masking operator. By reducing or eliminating the masking of the implant, it reduces or eliminates the operator error, resulting in higher percentages of acceptable finished products. Also, because the operator error from masking is eliminated or reduced, the quality of the finished products is also improved. The post-coating machining can reduce the amount of implants having manufacturing defects that are scrapped or have to be reworked.

In previous coating methods, when an implant part is not coated properly or the implant is damaged during the coating process and cannot be repaired, the part is discarded or recycled. The time and cost of machining the implant up to the coating step is lost or wasted. In the post-coating machining method, however, the coating procedure is performed before machining. If the part is damaged or improperly coated during the coating process, any loss of money and time expended on a discarded part is limited to the initial forming and coating of the part. There is less waste by coating the implant part earlier in the manufacturing process.

In addition, the post-coasting machining method allows the mechanical integrity of the part to be maintained, such as during one or more of the preparation or coating steps, because detailed portions of the part, such as thin or intricate portions, can be machined into the part after the one or more of the preparation or coating steps. In contrast, in previous coating methods, detailed portions of a part, such as thin or intricate portions, can crack, or load bearing characteristics of one or more portions can degrade from being subjected to one or more preparation or coating steps.

The post-coating machining method is also more efficient and has a lower scrap rate on finished products than previous methods, which can result in lower costs per part and increased production rate. The reduced time and money wasted on a scrapped part and the reduced number of scrapped parts from reducing or eliminating the masking step can lower the average production cost of each implant. Further, by reducing or eliminating the manual masking step, which can be time consuming and is a bottleneck in the production process, the production time can be faster. Also, without a masking step the number of parts that can be made per run can be increased, optimizing production efficiencies.

The post-coating machining method also eliminates or reduces the amount of coating ingress and overhang, which can occur in the pre-coating masking method. In previous coating methods, the coating 56 creeps under the masked areas or hangs over the edges of the implant 50 beyond the masked areas, creating an erratic border between coated and uncoated areas, as shown for example in FIGS. 2-5. FIG. 2 illustrates an implant 50 with a top side 56 covered with a coating 62. The coating 62 has an uneven edge with a proximal side 52. FIG. 3 illustrates a distal side 54 of the implant 50 and an uneven edge of the coating 62 on the top side 56. FIG. 4 illustrates a lateral side 60 of the implant 50 and an uneven edge of the coating 62 on the top side 56. FIG. 5 illustrates an uneven edge of the coating 62 on the bottom side 58 of the implant 50. The erratic border edges of the coating material can lead to areas of undesirable fusion and bone growth, which can result in conditions such as spinal stenosis, bone spurring, and the like. In some situations, the coating ingress or overhang may break off and contaminate the surgical area, which can lead to pain, infection and other complications.

In the post-coating machining method, any coating ingress onto areas not intended to be coated can be cut back during the machining process. The machining process following the coating process can produce a cleaner surface with a precise edge 164 between the coated surface and uncoated surface, as illustrated in FIGS. 7-10. FIG. 7 illustrates an implant 150 with a top side 156 covered with a coating 162. The coating 162 has a clean, precise edge 164 with a proximal side 152. FIG. 8 illustrates a distal side 154 of the implant 150 with a clean edge 164 of the coating 162 with the top side 156 and a clean edge 164 with the bottom side 158. FIG. 9 illustrates a lateral side 160 of the implant 150 and a clean edge 164 of the coating 162 on the top side 156 and the bottom side 158. FIG. 10 illustrates the bottom side 158 of the implant 150 covered with the coating 162. In the illustrated embodiments, the machined surface of the coating is flush with the surface of the implant, to produce a smooth continuous surface. The clean edge 164 can help limit fusion and bone growth to selected areas of the implant and help prevent conditions such as spinal stenosis and bone spurring. The clean edge 164 can be more aesthetically pleasing than a jagged edge produced by masking edges. In some embodiments, the machined surface of the coating comprises different surface morphologies or characteristics than the non-machined surface of the coating. In some embodiments, the machined surface of the coating comprises a more polished surface of the coating than the non-machined surface of the coating.

In previous coating methods, the masking method can leave a residue, adhesive, or contaminant on or within the surface of the implant when the masking is removed. In some situations, at least a portion of the residue, adhesive, or contaminant can be removed with solvent or additional processing steps, but this introduces additional chemicals and additional steps, thereby contributing additional time and cost to the overall process. If the residue, adhesive, or contaminant is not removed, it can infiltrate the surgical site, which can lead to pain, infection and other complications. The post-coating machining method, on the other hand, has reduced or eliminated masking, so residue, adhesive, or contaminant is eliminated or reduced from the process and implant. In some instances of the post-coating machining method, the machining process may remove the residue, adhesive, or contaminant, if any, during the post-coating machining steps.

Another advantage of the post-coating machining method is the ability to achieve tight dimensions for the coating and the implant, particularly for key features. As discussed earlier, masking before the coating procedure can result in coating ingress and imprecise edges. In some situations, the imprecise edges can cause misalignments between mating components, such as fasteners that are inserted at an incorrect angle because of coating that can interfere with the fastener path. In another example, coating ingress onto the mating surface of an implant can interfere with proper attachment of an insertion tool and can lead to improper positioning of the implant in the patient.

In the post-coating machining method, the edges are cleaner and more precise and are less likely to interfere with other components. The mating surfaces and other surfaces of the implant are also smoother and cleaner which allows better mating with other implants and tools. Key features of the implant can be coated and then machined to tight tolerances for improved cooperation with other components, whereas previously, coating the key features after machining could lead to deviations in the critical dimensions of the key features.

Although certain embodiments, features, and examples have been described herein, it will be understood by those skilled in the art that many aspects of the methods and devices illustrated and described in the present disclosure may be differently combined and/or modified to form still further embodiments. For example, any one component of the device illustrated and described above can be used alone or with other components without departing from the spirit of the present disclosure. Additionally, it will be recognized that the methods described herein may be practiced in different sequences and/or with additional devices as desired. Such alternative embodiments and/or uses of the methods and devices described above and obvious modifications and equivalents thereof are intended to be included within the scope of the present disclosure. Thus, it is intended that the scope of the present disclosure should not be limited by the particular embodiments described above, but should be determined only by a fair reading of the claims that follow.

What is claimed is:

1. A method of manufacturing an intervertebral implant comprising:
forming the implant with at least one dimension that is greater than a desired dimension, the implant comprising a superior bone facing surface, an inferior bone facing surface, a distal side, a proximal side, and lateral sides;
coating at least the superior bone facing surface and the inferior bone facing surface with an osteophilic material; and
machining one or more of the distal side, proximal side and lateral sides to the desired dimensions after coating;
wherein edges of the coating on the superior bone facing surface and the inferior bone facing surface are machined to be flush with the distal side, proximal side and/or lateral sides.

2. A method of manufacturing an orthopedic implant comprising:
forming a part with at least one dimension that is greater than a desired dimension;
coating at least a portion of the part with an osteophilic material; and
machining the part to the desired dimension after coating.

3. The method of claim 2, wherein machining the part to the desired dimension comprises machining portions of the coating.

4. The method of claim 3, wherein the coating is machined to be flush with a side of the part.

5. The method of claim 2, wherein the part is machined to the desired dimension by one or more of milling, drilling, laser cutting, electrical discharge machining, sanding, and filing.

6. The method of claim 2, wherein the osteophilic material is one of the group consisting of titanium, titanium alloy, cobalt-chrome, stainless steel, hydroxylapatite, and allograft.

7. The method of claim 2, further comprising assembling the part with a second part after machining.

8. The method of claim 2, further comprising assembling the part with a second part before machining.

9. The method of claim 2, wherein the part is formed by one or more of 3-D printing, molding, machining, casting, and extruding.

10. The method of claim 2, wherein the part is not masked before the coating step.

11. The method of claim 2, wherein machining the part comprises machining a tool engagement side of the implant.

12. A method of manufacturing an orthopedic implant comprising:
forming a first part with at least one dimension that is greater than a desired dimension;
coating at least a portion of the first part with an osteophilic material;
machining the first part to the desired dimension after coating;
forming a second part with at least one dimension that is greater than a desired dimension;
coating at least a portion of the second part with an osteophilic material;
machining the second part to the desired dimension after coating;
assembling together the first part and the second part.

13. The method of claim 12, wherein the orthopedic implant is an expandable intervertebral device.

14. The method of claim 12, wherein the first part is a top plate that is coated on a superior bone facing side and the second part is a bottom plate that is coated on an inferior bone facing side.

15. The method of claim 12, wherein machining the first part or second part to the desired dimension comprises machining portions of the coating.

16. The method of claim 15, wherein the coating is machined to be flush with a side of the first part or second part.

17. The method of claim 12, wherein the first part and the second part are machined to the desired dimension by one or more of milling, drilling, laser cutting, electrical discharge machining, sanding, and filing.

18. The method of claim 12, wherein the osteophilic material is one of the group consisting of titanium, titanium alloy, cobalt-chrome, stainless steel, hydroxylapatite, and allograft.

19. The method of claim 12, wherein the first part and the second part are formed by one or more of 3-D printing, molding, machining, casting, and extruding.

20. The method of claim 12, wherein the first part and the second part are not masked before the coating step.
